(19) United States
(12) Patent Application Publication
(10) Pub. No.: US 2025/0339189 A1
(43) Pub. Date: Nov. 6, 2025

(54) DEVICE FOR TREATING A FRACTURE

(71) Applicant: I.T.S. GMBH, Lassnitzhöhe (AT)

(72) Inventors: Volker BÜHREN, Murnau (DE); Ronald PRAGER, Bovenau (DE)

(21) Appl. No.: 18/834,343

(22) PCT Filed: Jan. 18, 2023
(86) PCT No.: PCT/AT2023/060009

§ 371 Date: Jul. 23, 2025

(30) Foreign Application Priority Data
Feb. 1, 2022 (AT) A 50051/2022

(51) Int. Cl. A61B 17/80; A61B 17/86; A61B 17/88

(52) U.S. Cl. CPC A61B 17/8061; A61B 17/8057; A61B 17/8625; A61B 17/8833

(57) ABSTRACT

The invention relates to a device (1) for treating a fracture, in particular a fracture of a proximal femur such as, for example, a femoral neck fracture, having a plate (3) for attaching to a thigh bone, one or more intraosseous screws (10) by means of which the plate (3), through intraosseous screw openings (14), can be fixed to a thigh shaft (4), a coupling device, in particular a bone screw (5), which is arranged proximally to the at least one intraosseous screw (10) and by means of which the plate (3) can be connected at a coupling device opening (6) through the thigh shaft (4) and a thigh neck (2) to a thigh head (7), wherein the coupling device is connected to the plate (3) with play (21) such that the coupling device can be moved in the lateral direction (8) relative to the plate (3). In order to achieve a particularly precise and simultaneous adjustability of the play (21), according to the invention an adjusting device is provided which is connected to the plate (3), which can be positioned at different positions relative to the plate (3) and by means of which the play (21) can be adjusted.

The invention relates to a device for treating a fracture, in particular a fracture of a proximal femur such as, for example, a femoral neck fracture, having a plate for attaching to a thigh bone, one or more intraosseous screws by means of which the plate, through intraosseous screw openings, can be fixed to a thigh shaft, a coupling device, in particular a bone screw, which is arranged proximally to the at least one intraosseous screw and by means of which the plate can be connected at a coupling device opening through the thigh shaft and a thigh neck to a thigh head, wherein the coupling device is connected to the plate with play such that the coupling device can be moved in the lateral direction relative to the plate.

Devices of the kind described in the introduction for treating a fracture of a femoral neck by connecting one side of the bone plate to the thigh shaft using the intraosseous screws, after which the thigh head is connected to the plate via the coupling device, which is usually designed as a bone screw, are known from the prior art. In this connection, a movable connection between the coupling device and the plate is advantageous for encouraging a healing process. Accordingly, it has been found that small movements and compression at a contact area between the parts of the bone fracture, that mis to say typically at a contact area in the region of the femoral neck, are advantageous for assisting the healing process, although at the same time it is important to prevent the bone parts from sliding into each other too much.

However, the devices known from the prior art, which in particular make use of “barrel” and/or “telescopic” screws and are known for example from WO 2007/109302 A2, have proven to be awkward.

This is where the invention comes into play. The object of the invention is to suggest a device of the kind described in the introduction, which is not only simple to manipulate but also particularly advantageous for a healing process.

This object is solved according to the invention by a device of the kind described in the introduction, in which an adjusting device is provided that is connected to the plate and placeable in various positions relative to the plate to adjust play.

Due to the inventive design of the device, it is thus no longer necessary to use telescopic screws or the like, but it was found in the context of the invention that a robust structure and at the same time simple method for adjusting play can be obtained simply with a corresponding adjusting device instead. At the same time, the adjusting device may be designed in a wide variety of ways, in order to affect a magnitude of play within which the coupling device is movable relative to the plate.

It is advantageous if the adjusting device is connected to the plate substantially rigidly in the lateral direction and the medial direction. This may be achieved for example with an adjusting device consisting of an adjusting screw that can be screwed into the plate, and by means of which a movability of the coupling device relative to the plate may be adjusted. The play of the coupling device relative to the plate may be adjusted by means of a screw-in depth of the adjusting screw.

It is particularly advantageously provided that the coupling device has a first stop surface that cooperates with a corresponding surface on the adjusting device and/or the plate, to such effect that a movability of the coupling device relative to the plate in medial direction is limited by the first stop surface. The coupling device may thus be moved in medial direction relative to the plate until the device bears on the first stop surface, so that a medial end position of the coupling device relative to the plate is defined by the first stop surface.

In principle, this first stop surface may be designed in any way, which bears on the adjusting device and/or the plate in medial direction after a predefined position of the coupling device, thereby preventing any further movement in medial direction. The stop surface may thus be aligned either normally to a longitudinal axis of the coupling device, along which said coupling device is movable relative to the plate, or at an angle to the longitudinal axis, in order to achieve a particularly low surface pressure.

For example, it may be provided that the coupling device has a collar laterally which cooperates with the plate to form a limit stop, up to which the coupling device may be advanced into the plate medially. The coupling device may then be used simply as a traction bolt, which pulls the thigh head against the plate. The coupling device then has a collar or shoulder at one lateral end, for example, which is designed to be larger than the coupling device opening, with the result that the coupling device can only be advanced into the plate as far as the collar in medial direction.

It is advantageous if the coupling device has a second stop surface, which bears on the adjusting device when the coupling device is in a lateral end position. In this way, play may be altered simply by changing the position of the adjusting device relative to the plate, since this causes a change in the distance between the stops.

The second stop surface may be formed by just a single surface, which in the lateral end position provides a contact between coupling device and adjusting device. However, it may also be provided that the second stop surface is formed by a plurality of surfaces, which function in parallel and so limit movability particularly effectively. Accordingly, it has been found that when only a single second stop surface is used, in particular bearing on a medial thread end of the adjusting device, there is a risk that the adjusting device may slide along the coupling device and/or screw itself onto the second stop surface on the coupling device via the thread, with the result that movability is less limited than intended. In particular, it may be provided that the second stop surface is formed by two or three limit stops or contact surfaces functioning in parallel, for example by a primary, a secondary and a tertiary second limit stop. In such a case, the primary second limit stop may bear on a lateral end of the adjusting device, for example, the secondary second limit stop may bear on the middle of the adjusting device, for example, and the tertiary second limit stop may bear on the adjusting device in the region of a medial end of the adjusting device.

It is advantageous if at least a part of the second stop surface is formed by a lateral second stop surface which in the lateral end position at least partly bears on a shoulder or collar of the adjusting device, in particular on an adjusting screw collar, in a region of the lateral end of the adjusting device. The collar, which is preferably designed as an adjusting screw collar, may be arranged on a lateral end of the adjusting screw, and may have a larger diameter than the other adjusting device, such that in the lateral end position the coupling device bears on the adjusting device, in fact on a lateral end of the adjusting device. In this case, the second stop surface on the coupling device may be arranged on a terminal, lateral circumferential depression to obtain a corresponding form lock with the collar on the adjusting device.

It is preferably provided that at least part of the second stop surface is formed by a medial second stop surface on the coupling device, at least part of which in the lateral end position bears on a thread of the adjusting device. In particular, a medial end of the thread on the adjusting device may thus serve as a limit stop for a corresponding surface of the coupling device in order to limit a movability of the coupling device in lateral direction.

It has been found to be advantageous if the adjusting device is connected to the plate by a thread, wherein a screw lock, in particular a plastic part is provided, which increases friction in the thread to prevent the adjusting device from becoming detached. In this simple manner, unintended detachment of the adjusting device from the plate is prevented.

In order to provide a simple way to prevent mistakes when implanting the device due to screwing it in too far, it is preferably provided that the adjusting device can only be screwed into the plate as far as a predefined position, this being realised in particular by design with a wrench, preferably an Allen wrench, which no longer produces a contact with a corresponding counterpart, in particular an internal hex in the adjusting device, beyond a predefined position of the adjusting device.

It is advantageous if the coupling device is movable between limit stops between a medial end position and a lateral end position, wherein the medial end position is defined by a first stop surface on the coupling device in cooperation with the adjusting device, and the lateral end position is defined by a second stop surface in cooperation with the adjusting device. The adjusting device thus simply defines the limit stops that limit a movability of the coupling device. Thus, both a position of the end positions and play may be adjusted in simple manner.

It has been found to be advantageous that the first stop surface and the second stop surface are arranged with a stop surface angle relative to one another, in particular at different angles to an adjustment direction, along which the adjusting device is movable relative to the plate, in particular with a stop surface angle relative to one another from 10 degrees to 160 degrees, preferably 60 degrees to 120 degrees, such that a movement of the adjusting device along the adjustment direction has differing effects on the lateral end position and the medial end position. Thus, not only a position of the limit stops, but also play, that is to say a movability of the coupling device between the limit stops may be altered by a change of position of the adjusting device. In particular, if a stop surface is approximately parallel to the adjustment direction, a position of the adjusting device along the adjustment direction does not change the limit stop formed by the corresponding stop surface and/or the corresponding end position.

Thus, for example, if the medial end position is defined by a first stop surface in cooperation with the adjusting device, which first stop surface is approximately parallel to the adjustment direction, a change of position of the adjusting device along the adjustment direction does not change the medial end position.

It is advantageous if the second stop surface is approximately normal to the adjustment direction and the first stop surface is approximately parallel to the adjustment direction, wherein in particular the adjustment direction is aligned at an angle from 0.5 degrees to 15 degrees, preferably 0.8 degrees to 2 degrees, relative to a longitudinal axis of the coupling device. Thus, a movement of the adjusting device along the adjustment direction causes a displacement of the second limit stop, but not of the first limit stop, providing a simple way to alter the play.

It is particularly preferably provided that the adjusting device is embodied as an adjusting screw and the coupling device is embodied as a bone screw, wherein a longitudinal axis of the adjusting screw is aligned at an angle from 0.5 degrees to 15 degrees, in particular 0.8 degrees to 2 degrees, relative to a longitudinal axis of the bone screw. In this way, play may be altered particularly simply by an interaction between these two screws. At the same time, a robust, easily manageable design is created. The adjusting screw and the bone screw are usually arranged adjacent to one another and preferably protrude through a shared opening in the plate. This enables stop surfaces between adjusting screw and bone screw that limit play of the bone screw relative to the plate to be realised in a structurally simple and compact manner.

It has been found to be advantageous that the adjusting device is embodied as an adjusting screw, which is arranged in a thread in the plate and may be positioned variably by means of the thread. The thread is preferably self-locking. The adjusting device is thus movable in lateral direction and medial direction only by rotation, but not by pressure in the respective lateral direction or medial direction. In this way, the play can be adjusted particularly precisely.

Accordingly, the adjusting device is preferably positionable variably along an adjustment direction, in particular along a thread axis, relative to the plate.

It is particularly advantageous if the adjustment direction is aligned at an angle from 0.5 degrees to 10 degrees, in particular 0.8 degrees to 2 degrees relative to a longitudinal axis of the coupling device, wherein the adjustment direction and a longitudinal axis of the coupling device are preferably in the same plane. This enables a particularly precise adjustment capability of the play. A first stop surface that is in particular simple by design may then be realised, on which the coupling device in a medial end position bears on the adjusting device, which is aligned at a corresponding angle to a longitudinal direction of the coupling device, and on which the coupling device bears on the adjusting device, with the result that here a particularly low surface pressure is achieved because of the small angle. This ensures particularly good stability.

It is preferably provided that the coupling device has a shoulder, which in particular adjoins a beveled region, wherein the shoulder forms a second stop surface, which bears on the adjusting device in a lateral end position of the coupling device, in particular approximately at the frontal face of the adjusting device. In this way, play is defined clearly by means of both stop surfaces.

It is particularly advantageous if the two stop surfaces are arranged at different angles to an adjustment direction of the adjusting screw, along which adjustment direction the adjusting screw may be moved relative to the plate. In this way, a movement of the adjusting screw along the adjustment direction has different effects on a medial end position and a lateral end position of the coupling device, thereby enabling the play to be adjusted simply.

The adjusting device is preferably embodied as an adjusting screw, and therefore typically has an approximately cylindrical enveloping surface. With a corresponding beveled surface on the coupling element, this then produces a line contact between the adjusting device and the coupling device in the region of the beveled surface, which may function as a first stop surface, which is aligned at an angle, that is to say not normally, to a direction of movement of the coupling device along the longitudinal axis of the coupling device. This assures good force transmission at the first stop surface. If the second stop surface is positioned on a shoulder or a transition from the beveled region to a for example cylindrical region adjacent thereto, the second stop surface may be aligned approximately normally to the longitudinal axis of the coupling device, with the result that a movement of the adjusting device along the adjustment direction has effects of different strength on a position of the medial end position, which is typically produced by the first stop surface in cooperation with the adjusting device, and the lateral end position, which is typically produced by the second stop surface in cooperation with the adjusting device. In this way, the play may easily be altered changing a position of the adjusting device along the adjustment direction, even though both stop surfaces cooperate with the adjusting device.

The cylindrical region of the coupling device usually adjoins the conical region medially, so a shoulder is created between conical region and cylindrical region, which shoulder is usable as second stop surface, to limit the movability of the coupling device in lateral direction relative to the plate.

It is advantageous if the coupling device has a beveled region and the adjusting device has an outer contour at least a region of which is cylindrical, wherein the beveled region forms a first stop surface on the coupling device, which in cooperation with the approximately cylindrical outer contour of the adjusting device limits a movability of the coupling device relative to the plate in medial direction. A cylindrical outer contour of the adjusting device is obtained for example when the adjusting device is formed by an adjusting screw. This assures a particularly effective transmission of force between coupling device and adjusting device and enables a limit stop that can be adjusted easily and robustly in order to define play.

It is particularly preferably provided that the beveled region is aligned at a bevel angle to a longitudinal axis of the coupling device, which angle corresponds to an angle at which the adjusting device is aligned with the longitudinal axis of the coupling device, and the adjusting device is arranged in such manner that the adjusting device touches the beveled region. The adjusting device may then be screwed into the plate along the adjustment direction without colliding with the coupling device, so that a position of a lateral end position is easily changeable. A medial end position is then typically obtained from dimensions of adjusting device and coupling device and may be independent of a position of the adjusting device along the adjustment direction.

It is advantageous if a traction bolt is provided, which is arranged approximately parallel to a longitudinal axis of the coupling device, in particular proximal to the coupling device, with which traction bolt the plate at a traction bolt opening may be connected to the thigh head through the thigh shaft and a femoral neck, to prevent a rotation of the thigh head relative to the coupling device.

It is usually provided that a longitudinal axis of the coupling device is aligned at a femoral neck angle from 10 degrees to 60 degrees, in particular 35 degrees to 45 degrees, relative to a longitudinal axis of the first screw. The corresponding angles are obtained by arranging and aligning the coupling device opening and the first openings in the plate, and are then optimal for treating femoral neck fractures, since an angle between a femoral neck and a thigh shaft is usually in the region of about 130 degrees.

It is preferably provided that the coupling device is of hollow construction, so that bone cement can be introduced through the coupling device in the region of the thigh head when the device is arranged on a thigh bone.

It is also preferable if the traction bolt is of hollow construction, so that bone cement can be introduced into the region of the thigh head through the bolt as well if necessary.

Further features, advantages and effects of the invention are discernible with reference to the following description of an exemplary embodiment. In the drawing, to which reference will be made, the figures show:

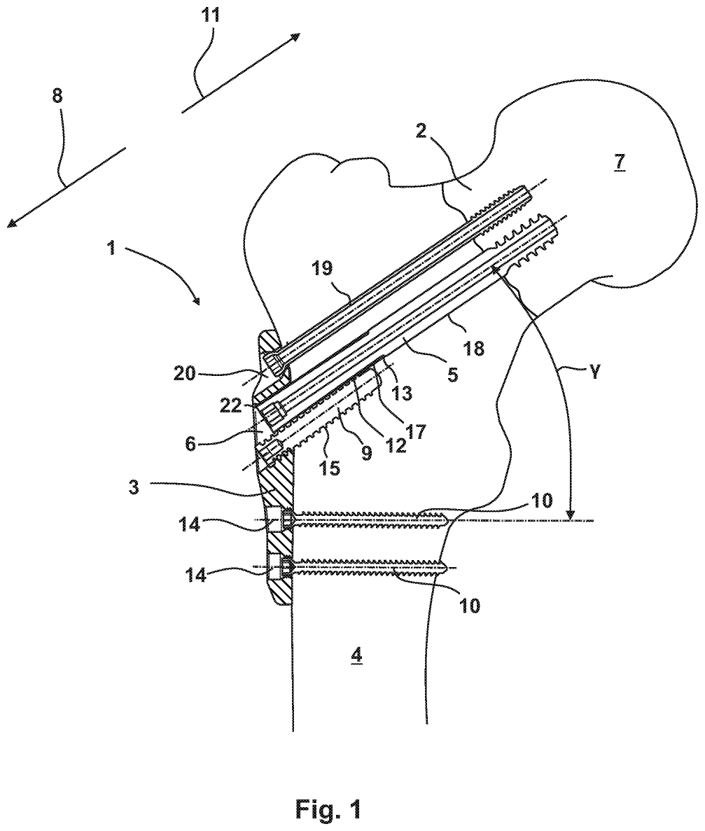

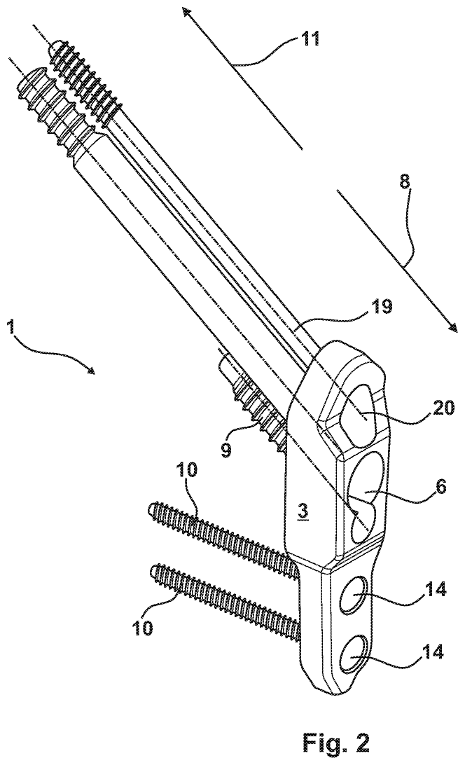

FIGS. 1 and 2 various views of a device according to the invention;

FIG. 2a a detail from FIG. 1;

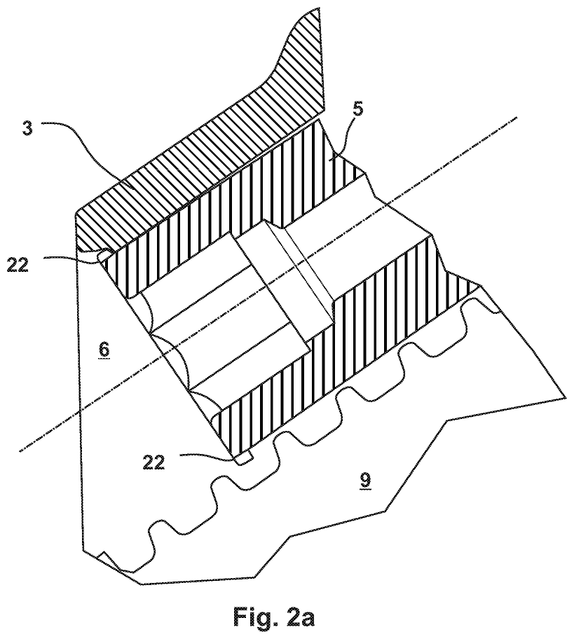

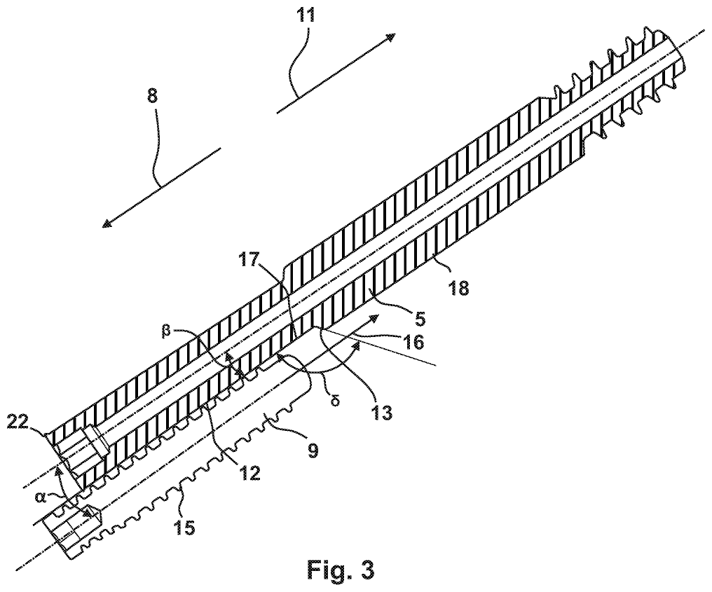

FIGS. 3 and 4 a detail of the device in various operating states;

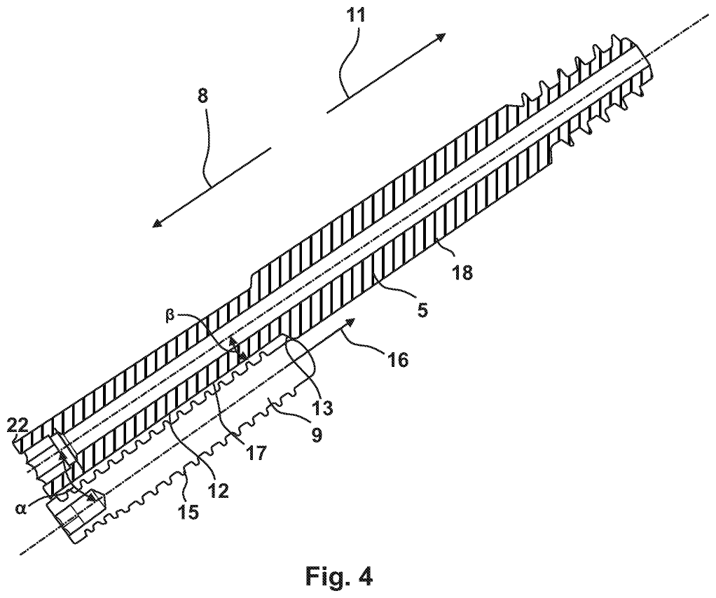

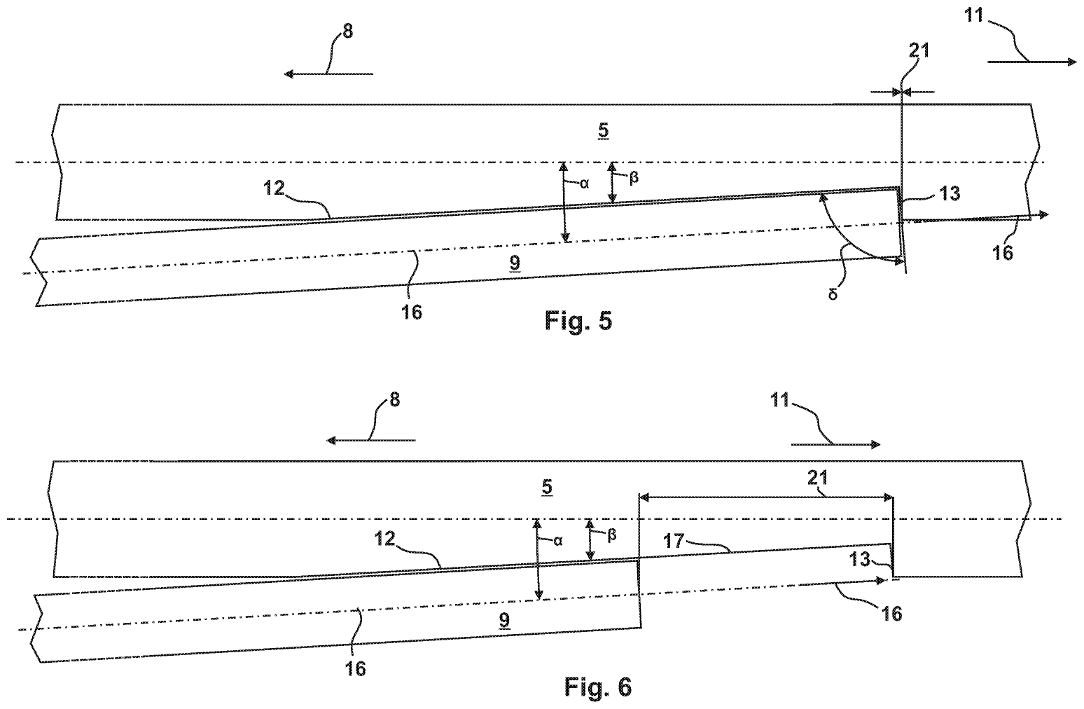

FIGS. 5 and 6 representations illustrating the operating principle of the device;

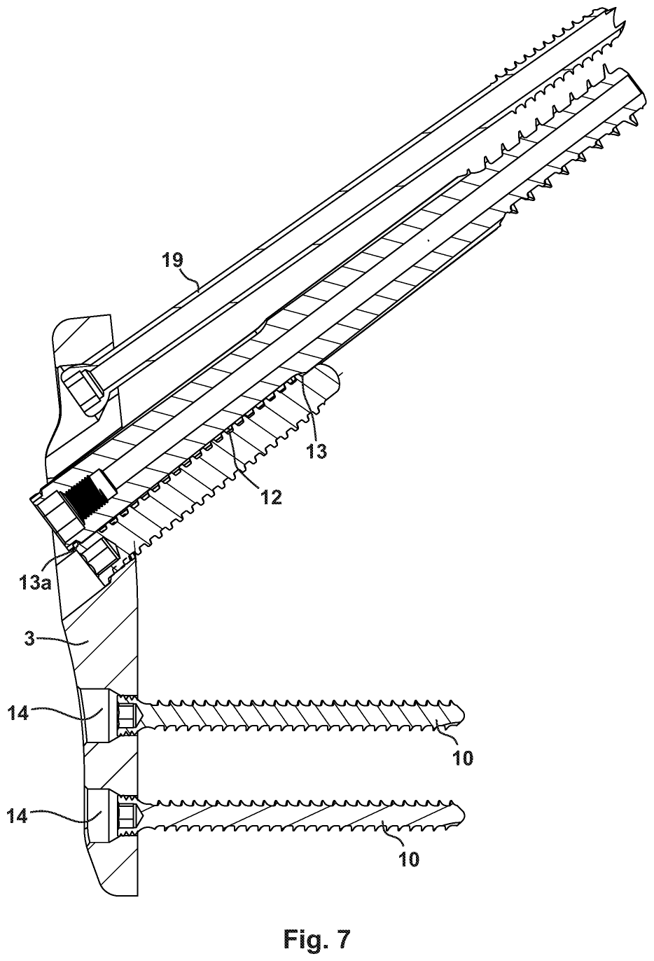

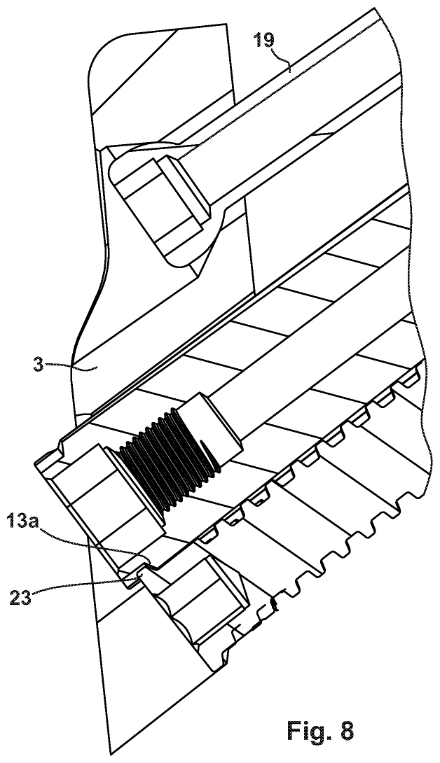

FIGS. 7 and 8 details of a further device according to the invention.

FIG. 1 shows a cross-sectional representation of a device 1 according to the invention on a thigh bone with a thigh shaft 4, a femoral neck 2 and a thigh head 7. In FIG. 2, this device 1 is represented in a 3D view without the thigh bone.

In each case, it may be seen that a plate 3 has been provided, which may be attached to the outside of a thigh bone, opposite a femoral neck 2, and may be connected to the thigh shaft 4 by intraosseous screws 10 that protrude through intraosseous screw openings 14 in a lower region of the plate 3.

A coupling device, here in the form of a bone screw 5, and a traction bolt 19, are provided above the intraosseous screws 10 and proximal thereto and are embodied as shaft screws and only have a thread 15 in an end region thereof. This makes it possible to pull the thigh head 7, into which said screws are screwed, against the femoral neck 2 and the thigh shaft 4, and thus achieve stabilisation of the fracture. For this purpose, in the plate 3 there are provided a coupling device opening 6, through which the bone screw 5 protrudes, and a traction bolt opening 20, at which the traction bolt 19 passes through the plate 3. The bone screw has a collar 22 at a lateral end, which collar bears on the coupling device opening 6 and thus forms a limit stop, up to which the bone screw can be introduced medially into the plate.

The bone screw 5 is connected to the plate 3 with play 21 in medial direction 11 and lateral direction 8. The play 21 is enabled by a movability of the coupling device relative to the plate 3 between a first limit stop and a second limit stop, each of which is located on an adjusting device that is formed here by an adjusting screw 9. The adjusting screw 9 has a thread 15, which corresponds to a thread 15 in the plate 3, so that the adjusting screw 9 can be either screwed into the plate 3 along an adjustment direction 16, approximately in medial direction 11, or in the opposite direction, approximately in lateral direction 8, out of the plate 3, to influence play 21 of the bone screw 5 relative to the plate 3, and therewith also a movability of the thigh head 7 relative to the femoral neck 2.

The first limit stop is formed by a first stop surface 12 in a beveled region 17 on the bone screw 5, which first stop surface 12 bears circumferentially on the adjusting screw 9 in a medial end position of the bone screw 5. Thus, the adjustment direction 16 is not parallel to a longitudinal axis of the bone screw 5 but is arranged at an angle a of about 1.2 degrees relative thereto. The beveled region 17 on the bone screw 5 is approximately parallel to the adjustment direction 16, which means that a normal to the beveled region 17 is approximately normal to the adjustment direction 16, and consequently the medial end position is independent of a screw-in depth of the adjusting screw 9. Moreover, the bone screw 5 in the medial end position bears on the plate 3 via the collar 22 provided at the lateral end, with the result that this collar 22 too forms a limit stop and defines the medial end position.

The second limit stop is formed by a second stop surface 13 in a region of a shoulder of the bone screw 5, which is created in a region between the beveled region 17 and a cylindrical region 18 of the bone screw 5. When the bone screw 5 is in a lateral end position, said shoulder bears on the frontal face of the adjusting screw 9. The lateral end position thus varies along the adjustment direction 16 with the screw-in depth of the adjusting screw 9. In this way, a change in the position of the adjusting screw 9 relative to the plate 3 leads to a change in the lateral end position of the bone screw 5 and consequently a change in the play 21.

A further traction bolt 19 is provided proximally to the bone screw 5 and is also screwed from the plate 3 into the thigh head. This additional traction bolt 19 both stabilises the thigh head 7 at the femoral neck 2 and prevents a rotation of the thigh head 7 about the bone screw 5 and/or the coupling device.

As is shown, the bone screw 5 and the traction bolt 19 are aligned approximately parallel to and at a femoral neck angle Y of about 20 degrees to 40 degrees relative to the intraosseous screws 10.

FIG. 2a shows an enlarged detail of FIG. 1. Here, the circumferential collar 22 in particular is clearly evident, and bears on the plate when the bone screw 5 is in the medial end position, thus forming a limit stop, up to which the bone screw 5 can be advanced into the plate 3. As is evident, the coupling device opening 6 is smaller than the collar 22, but large enough to ensure that the bone screw 5 can be advanced through the coupling device opening 6 into the plate 3 as far as the collar 22.

FIGS. 3 and 4 show the bone screw 5 and the adjusting screw 9 of the device 1 represented in FIGS. 1 and 2 in detail. FIG. 3 shows the bone screw 5 and the adjusting screw 9 with the bone screw 5 in a medial end position, which is to say when the distance between the thigh head 7 and the femoral neck 2 is greatest. As may be seen, no further movement of the bone screw 5 in medial direction is then possible, as the bone screw 5 is bearing on the beveled region 17 that forms the first stop surface 12, on a circumferential region of the adjusting screw 9. However, it would be possible to move the bone screw 5 and the thigh head 7, into which the end of the bone screw 5 is normally screwed, out of this position in the opposite direction, which is to say in lateral direction 8.

As is shown clearly here, the first stop surface 12 and the second stop surface 13 are arranged at a stop surface angle δ of about 110 degrees relative to one another. In this context, the first stop surface 12 is orientated approximately parallel to the adjustment direction 16, and accordingly the second stop surface 13 is orientated at an angle α of about 110 degrees relative to the adjustment direction 16. Consequently, a change of a position of the adjusting screw 9 along the adjustment direction 16 only causes a change to the lateral end position, but no change to the medial end position, which is also determined by the collar 22.

FIG. 4 shows the bone screw 5 and the adjusting screw 9 in a lateral end position of the bone screw 5, that is to say when the thigh head 7 cannot be moved closer to the femoral neck 2, and consequently no further movement of the bone screw 5 in lateral direction is possible. As may be seen, the bone screw 5 in this lateral end position bears on a second stop surface 13 formed by a shoulder on the bone screw 5 approximately at the frontal face on the adjusting screw 9.

A bevel angle β, at which the beveled region 17 is aligned relative to a longitudinal axis of the bone screw 5, corresponds approximately to an angle α between the adjustment direction 16 and the longitudinal axis of the bone screw, so the beveled region 17 is approximately parallel to the adjustment direction 16. Consequently, a change to the screw-in depth of the adjusting screw 9 has no effect on a position of the medial end position. Instead, this position is determined solely by dimensions of bone screw 5 and adjusting screw 9 and the angle α. However, a position of the lateral end position does change with the screw-in depth of the adjusting screw 9, since this is determined by a position of a front face of the adjusting screw 9. Thus, play 21 by which the bone screw 5 is movable relative to the plate 3 may be easily adjusted by a change of the screw-in depth of the adjusting screw 9 via the lateral end position. This adjustability is particularly important for rapid healing of a corresponding fracture.

As may be seen in FIGS. 1, 3 and 4, the bone screw 5 is of hollow design, so that bone cement can be introduced through it into a region of a thigh head 7 in order to fix the bone screw 5 there, for example.

In this context, FIGS. 5 and 6 show an effect of a screw-in depth of the adjusting screw 9 on the play 21, wherein for purposes of clarity dimensions and angle α are not shown to scale. As may be seen, the first stop surface 12 and a medial end position is not changed along the adjustment direction 16 with a screw-in depth of the adjusting screw 9 due to the beveled region 17 of the bone screw 5 approximately parallel to the adjustment direction 16, but the screw-in depth has direct effects on the second stop surface 13, which defines a the lateral end position. Accordingly, FIG. 5 shows situation in which the adjusting screw 9 has been screwed substantially into the plate 3 (not shown), so that there is no play 21 left. In this situation, the bone screw 5 bears on both the first stop surface 12 and the second stop surface 13. In the situation illustrated in FIG. 6, the adjusting screw 9 is arranged laterally further outwards, or is not screwed as far into plate 3 as is shown in FIG. 5.

The adjusting device embodied as adjusting screw 9 is aligned at an angle α of about 1.2 degrees relative to the longitudinal axis of the bone screw 5. A cone angle of the conical region is also designed correspondingly, so that a line contact is produced between the adjusting screw 9 and the conical region of the bone screw 5, thereby assuring particularly good stabilisation and force transmission. Here too, it may be seen that the first stop surface 12 is aligned at a stop surface angle δ relative to the second stop surface 13, which angle in this case is equal to about 90 degrees, with the result that the effects of a change of position of the adjusting screw 9 are different for the medial end position and the lateral end position.

Although in the embodiments a corner or edge is illustrated at a connection between the first stop surface 12 and the second stop surface 13, it is clear that the first stop surface 12 and the second stop surface 13 could also be connected by a rounding, for example, and might still have a corresponding stop surface angle δ relative to one another. The bone screw 5 may therefore be produced easily using either a ball nose cutter, for example, or a shank type milling cutter or the like.

FIGS. 7 and 8 show details of a further exemplary embodiment of a device 1 according to the invention. This device 1 has a second stop surface 13 formed by two contact surfaces, namely a primary second stop surface 13 on the coupling device, which bears on a medial thread end of the adjusting device in the lateral end position, and a secondary second stop surface 13a on a lateral end of the adjusting device, which operate in parallel, wherein the secondary second stop surface 13a as shown in the detail of FIG. 8 is formed by an adjusting screw collar 23, that is to say a region of the adjusting screw 9 having a larger diameter than the rest of the adjusting screw 9, in conjunction with a frontal tapering and/or circumferential depression in the coupling device, in this case the bone screw 5. The effect of the two second stop surfaces 13, 13a, which function in parallel, provides a simple way to prevent the unintended sliding of the adjusting screw 9 along the coupling device, and this also reliably limit movability.

A device 1 according to the invention enables a fracture of a thigh bone to be treated particularly simply and ensures particularly effective healing due to the easily adjustable play 21.

1. Device for treating a fracture, having a plate for attaching to a thigh bone, one or more intraosseous screws by means of which the plate, through intraosseous screw openings, can be fixed to a thigh shaft, a coupling device, which is arranged proximally to the at least one intraosseous screw and by means of which the plate can be connected at a coupling device opening through the thigh shaft and a thigh neck to a thigh head, wherein the coupling device is connected to the plate with play such that the coupling device can be moved in the lateral direction relative to the plate, wherein an adjusting device is provided which is connected to the plate, and which can be positioned at different positions relative to the plate and by means of which the play can be adjusted.

2. Device according to claim 1, wherein the adjusting device is connected to the plate substantially rigidly in the lateral direction and the medial direction.

3. Device according to claim 1, wherein the coupling device has a first stop surface, that cooperates with a corresponding surface on the adjusting device and/or the plate, to such effect that a movability of the coupling device relative to the plate in medial direction is limited by a first stop surface.

4. Device according to claim 1, wherein the coupling device has a collar laterally which cooperates with the plate to form a limit stop, up to which the coupling device may be advanced into the plate medially.

5. Device according to claim 1, wherein the coupling device has a second stop surface, which bears on the adjusting device when the coupling device is in a lateral end position.

5. Device according to claim 5, wherein at least a part of the second stop surface is formed by a lateral second stop surface which in the lateral end position at least partly bears on a shoulder or collar of the adjusting device, in a region of the lateral end of the adjusting device.

7. Device according to claim 5, wherein at least a part of the second stop surface is formed by a medial second stop surface on the coupling device, which at least partly bears on a thread of the adjusting device in the lateral end position.

8. Device according to claim 1, wherein the adjusting device is connected to the plate by a thread, wherein a screw lock, is provided, which increases friction in the thread to prevent the adjusting device from becoming detached.

9. Device according to claim 1, wherein the adjusting device can only be screwed into the plate as far as a predefined position, wherein this is realised which no longer produces a contact with a corresponding counterpart, beyond a predefined position of the adjusting device.

10. Device according to claim 1, wherein the coupling device is movable between limit stops between a medial end position and a lateral end position, wherein the medial end position is defined by a first stop surface on the coupling device in cooperation with the adjusting device, and the lateral end position is defined by a second stop surface in cooperation with the adjusting device.

11. Device according to claim 1, wherein the first stop surface and the second stop surface are arranged at a stop surface angle (δ) relative to one another, along which the adjusting device is movable relative to the plate, so that a movement of the adjusting device along the adjustment direction has differing effects on the lateral end position and the medial end position.

12. Device according to claim 11, wherein the second stop surface is approximately normal to the adjustment direction, and the first stop surface is approximately parallel to the adjustment direction.

13. Device according to claim 1, wherein the adjusting device is embodied as adjusting screw and the coupling device is embodied as bone screw, wherein a longitudinal axis of the adjusting screw is aligned at an angle (α) from 0.5 degrees to 15 degrees, to a longitudinal axis of the bone screw.

14. Device according to claim 1, wherein the adjusting device is embodied as adjusting screw, which is arranged in a thread in the plate and may be positioned variably by means of the thread.

15. Device according to claim 1, wherein the adjusting device is positionable variably along an adjustment direction, relative to the plate.

16. Device according to claim 15, wherein the adjustment direction is aligned at an angle (α) from 0.5 degrees to 10 degrees, relative to a longitudinal axis of the coupling device, wherein the adjustment direction and a longitudinal axis of the coupling device are in one plane.

17. Device according to claim 1, wherein the coupling device has a shoulder, wherein the shoulder forms a second stop surface, which bears on the adjusting device in a lateral end position of the coupling device.

18. Device according to claim 1, wherein the coupling device has a beveled region, and at least a region of the adjusting device has an approximately cylindrical outer contour, wherein the beveled region on the coupling device forms a first stop surface, which in cooperation with the outer contour of the adjusting device limits a movability of the coupling device relative to the plate in medial direction.

19. Device according to claim 18, wherein the beveled region is aligned at a bevel angle (β) to a longitudinal axis of the coupling device, which angle corresponds to an angle (α) at which the adjusting device is aligned with the longitudinal axis of the coupling device, and the adjusting device is arranged in such manner that the adjusting device touches the beveled region.

20. Device according to claim 1, wherein a traction bolt is provided, which is arranged approximately parallel to a longitudinal axis of the coupling device, with which traction bolt the plate at a traction bolt opening can be connected to the thigh head through the thigh shaft and a femoral neck, to prevent a rotation of the thigh head relative to the coupling device.

21. Device according to claim 1, wherein a longitudinal axis of the coupling device is aligned at a femoral neck angle (γ) from 10 degrees to 60 degrees, relative to a longitudinal axis of the intraosseous screw.

22. Device according to claim 1, wherein the coupling device is of hollow design, so that bone cement can be introduced into the region of the thigh head through the coupling device when the device is arranged on a thigh bone.
